(19) United States
(12) Patent Application Publication
(10) Pub. No.: US 2019/0282260 A1
(43) Pub. Date: Sep. 19, 2019

(54) APPARATUS WITH NEEDLE FOR THE EXTRACTION OF HAIR FOLLICLES IN LONG FORM

(71) Applicant: ASMED OZEL SAC EKIMI SAGLIK HIZMETLERI TICARET A.S., ATASEHIR-ISTANBUL (TR)

(72) Inventor: Koray ERDOGAN, Atasehir/Istanbul (TR)

(21) Appl. No.: 16/347,499

(22) PCT Filed: Nov. 6, 2017
(86) PCT No.: PCT/TR2017/050549

§ 371 Date: May 3, 2019

(30) Foreign Application Priority Data
Nov. 5, 2016 (TR) 2016/15812

(51) Int. Cl. A61B 17/3205

(52) U.S. Cl. CPC A61B 17/32053; A61B 2017/320064

(57) ABSTRACT

The invention relates to a needle that enables the extraction of hair follicles for hair transplantation without the need for a prior trimming of the hair.

### TECHNICAL FIELD

The invention relates to an apparatus with a needle, that enables the extraction of hair follicles before the hair transplantation without the need for a prior trimming of the hair.

### KNOWN STATUS OF THE TECHNIQUE

During embryogenesis, lifeless extensions, namely “hair (or bristle), develop in the follicle, which elongate the mesenchymal cells to the dermis or subcutaneous tissue in a convoluted way. In many parts of the human body there are hairs of different lengths.

In the head region of the human body there are hair root bulbs found under the skin named the scalp and that cannot be seen with the eye. With signals taken from certain parts of the body, the roots become active and the hair type called hair reaches the surface from under the scalp and grows by an average of 0.41 mm every day. In the head region of the human body there are an average of 100,000-125,000 hairs. Each hair has an independent life cycle. Considering this cycle, it is observed that an average of 100 hairs per day are shed.

Besides normal hair loss, the shedding of more hair and no appearance of new hair in people who are exposed to cases like stress, smoking, radiation, chemotherapy, various treatment methods, and genetic characters etc. is observed. Over time, hair may not be found in certain parts of the scalp or it is entirety. Hair growth can once again be provided with the hair follicle taken from the human body being transferring to the region where hair does not exist and does not come out again with the hair transplantation method.

Hair transplantation begins with the shaving of hair in the nape of the person for which the transplant will be done. The shortened hair is taken from its roots under a microscope. The area where hair is to be transplanted is opened with micro-blades. The hair follicles taken from the nape region are taken with tweezers and placed into the channels that are provided with hair follicles by being pushed in. This can be done with manual or motorized tools. The yield provided with the hair follicles placed by the pushing method holding by settling in the canal is 8 hair follicles in 10 hair follicles. That is, a loss of efficiency of 20% occurs. Since this process takes a long time, and is painful and costly, 20% inefficiency is a high level and the frequency and natural image desired to be given at the end of the process are not fully ensured. In addition, the healing of the channels opened on the scalp where the hair transplant was made and the growth of the hair can be as much as 1 year.

### Problems the Invention Aims to Solve and Analysis

The invention provides a natural, frequent and desirable look on the same day of the hair transplantation after the removal of the follicles with long hair and their transplantation on the scalp.

Before the invention technique, the hair transplant process starts with the shaving of the region where hair follicles are going to be taken as general use expressed as being a 0 or 1 number or in other words where the stand of hair is under 1 mm in length. It is completed with the hair follicles taken from this area being placed into the hairless zone to be treated in the channels opened with micro knives. After this process where a minimum of 2500 hair follicles are positioned, a negative appearance consisting of thousands of red, small wounds appears. The growth of strands of hair and the disappearance of this negative appearance can take approximately 12 months. After this application, effects on the psychology of the person due to this negative appearance occur and the person experiences problems in normal routine life. With the invention technique, it can ensure that the application is carried out by taking hair follicles without the region where the hair follicles to be taken being shaved. After the process carried out with the invention technique, the individual can continue with their daily life. The frequency and natural appearance desired to be given are provided on the same day.

### DISCLOSURE OF THE INVENTION VIA DRAWINGS

FIG. 1. Isometric side view of the invention

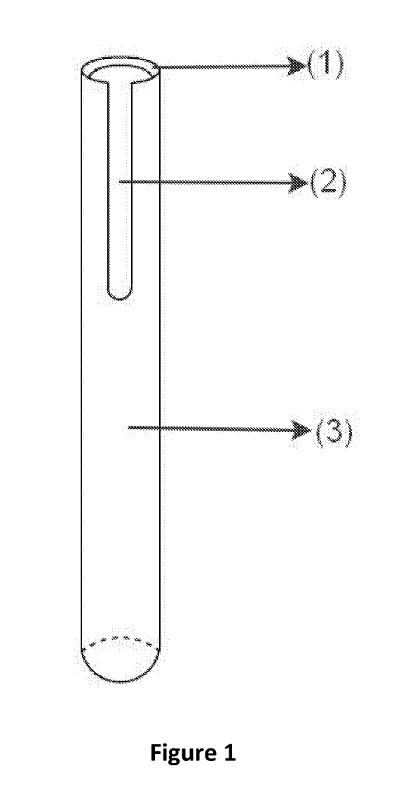

FIG. 2. View of the Invention During Usage

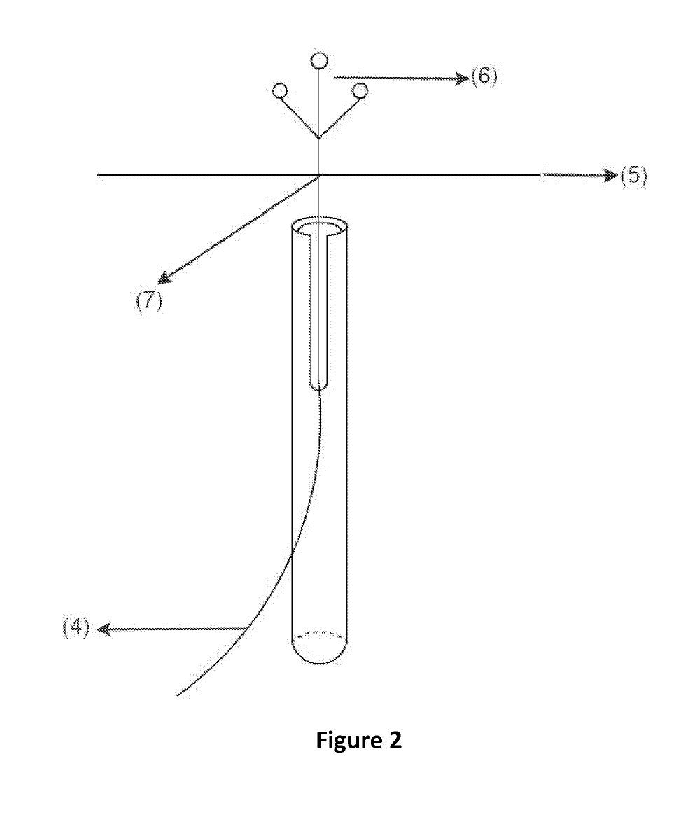

### NUMBERING OF THE SECTIONS

1. Sharp tip
2. Canal
3. Handle Area
4. Hair
5. Skin
6. Tissue
7. Hair Follicle

### TECHNICAL SPECIFICATIONS AND DESCRIPTION OF THE INVENTION

The invention relates to an apparatus with a needle that enables the extraction of hair follicles before the hair transplantation without the need for a prior trimming of the hair.

The invention is placed in a handle section at a depth determined by the practitioner of the application from the holding area (3). The sharp tip (1) is held on the skin by revealing the hair follicle (7) and allowing the hair (6) to enter the channel (2). The hand-held handle is rotated by circular movements until it reaches the desired depth. When the desired depth is reached, a cut is formed in the tissue (6) in the vicinity of the hair follicle (7), and an incision is made so that the hair (4) is excluded from the invention. The channel (2) on the holding section (3) keeps the hair (6) out of the invention and prevents it from being damaged without being subjected to the cutting process. After the cutting processing is finished, the invention is withdrawn and passed to the next hair follicle (7). By means of the channel 2, the non-damaged hair (4) is removed from the skin (5) by using a tweezer, together with the hair follicle (7) and tissue (6). It is then deposited in a container for transplantation in a bald area.

After the hair transplantation is complete, the person will be able obtain the desired long hair on the same day.

1- The invention relates to an apparatus with a needle that enables the extraction of hair follicles in a long form and is characterized in that; it consists of a sharp tip (1), canal (2), handle area (3), hair (4), skin (5), tissue (6) and hair follicle (7).

2- according to claim 1, the invention relates to an apparatus with a needle that enables the extraction of hair follicles in a long form and is characterized in that; it contains a canal (2) that holds the long hair (4).

3- according to claim 1, the invention relates to an apparatus with a needle that enables the extraction of hair follicles in a long form and is characterized in that; it contains a sharp tip that creates a cut on the skin (5).
